(19) United States
(12) Patent Application Publication
(10) Pub. No.: US 2016/0015338 A1
(43) Pub. Date: Jan. 21, 2016

(54) DOSIMETER

(71) Applicants: TORECK CO., LTD., Yokohama-shi, Kanagawa (JP); NATIONAL UNIVERSITY CORPORATION, TOHOKU UNIVERSITY, Sendai-shi, Miyagi (JP)

(72) Inventors: Koichi Chida, Miyagi (JP); Masayuki Zuguchi, Miyadi (JP); Masaski Nakamura, Miyagi (JP); Koetsu Sato, Kanagawa (JP); Tsutomu Iyoki, Kanagawa (JP); Eiichi Uchijima, Tokyo (JP)

(21) Appl. No.: 14/771,725

(22) PCT Filed: Feb. 26, 2014
(86) PCT No.: PCT/JP2014/054706

§ 371 Date: Aug. 31, 2015

(30) Foreign Application Priority Data
Mar. 6, 2013 (JP) 2013-044772

(51) Int. Cl. A61B 6/00; G01T 1/10; G01T 1/02

(52) U.S. Cl. CPC A61B 6/4216; A61B 6/485; G01T 1/023; G01T 1/10

(57) ABSTRACT

Dosimeters with an environmental safety are provided, by which a dose of radiation can be measured in real time when taking or fluoroscopically viewing an image by using the radiation while suppressing influence on the image taken or fluoroscopically viewed. The present dosimeter comprises a radiation detection part 100 including a phosphor 120 made of Y₂O₂S as a matrix with at least Eu as an activating agent, an optical fiber 200 of transmitting light emitted from the phosphor 120 of the radiation detection part 100 by receiving a radiation, and a light detection part 310 of detecting the light transmitted through the optical fiber 200.

### TECHNICAL FIELD

The present invention relates to dosimeters of measuring a dose of radiation such as an X-ray.

### BACKGROUND ART

A dosimeter is conventionally known (Non-Patent Literature 1), in which a cadmium (Cd) scintillator is used for a detection unit of detecting a radiation and light generated by the detection unit is transmitted through an optical fiber and detected. Since the detection unit and the optical fiber have a satisfactory transparency to an X-ray, this dosimeter is, for example, capable of measuring an absorption dose on a skin surface of a human body in real time while suppressing influence on an X-ray image when taking or fluoroscopically viewing the X-ray image. Therefore, the dosimeter has been widely used particularly in the field of medical image diagnosis.

### CITATION LIST

### Non-Patent Literature

Non-Patent Literature 1: Hwang E, Gaxiola E, Vlietstra R E, et al.: Real-time measurement of skin radiation during cardiac catheterization. Cathet Cardiovasc Diagn, 43 (4), pp. 367-370 (1998).

### SUMMARY OF INVENTION

### Technical Problem

However, the conventional dosimeter has an environmental problem because the scintillator used in the detection unit includes cadmium (Cd).

### Solution to Problem

A dosimeter according to an aspect of the present invention is a dosimeter of measuring a dose of radiation and comprises a radiation detection part including a phosphor made of Y₂O₂S as a matrix with at least Eu as an activating agent, an optical fiber of transmitting light emitted from the phosphor of the radiation detection part by receiving a radiation, and a light detection part of detecting the light transmitted through the optical fiber.

In this dosimeter, when the radiation detection part receives a radiation, the phosphor included in the radiation detection part emits light. The light emitted from the radiation detection part is entered into the optical fiber and transmitted. This light transmitted with the optical fiber is detected by the light detection part. The dose of radiation can be measured based on the detection result of the light detection part.

Herein, since the light that is emitted from the radiation detection part by receiving a radiation can be transmitted to the light detection part away from the radiation detection part by the optical fiber, the radiation is not blocked by the light detection part. Furtheremore, the foregoing phosphor made of Y₂O₂S as a matrix with at least Eu as an activating agent has a satisfactory transparency to the radiation and the optical fiber has also a satisfactory transparency to the radiation unlike a cable and lead wire made of normal metal. Accordingly, when taking or fluoroscopically viewing an image by using a radiation, it is capable of suppressing an influence on the image taken or fluoroscopically viewed. Therefore, it is capable of measuring a radiation dose in real time when taking or fluoroscopically viewing the image while suppressing influence on the image taken by using the radiation.

Moreover, since the phosphor in the radiation detection part is the phosphor made of Y₂O₂S as a matrix with at least Eu as an activating agent and does not include cadmium (Cd), it is capable of providing a dosimeter with an environmental safety.

In the foregoing dosimeter, the radiation may be an X-ray emitted from an X-ray generating apparatus with a tube voltage of 40 kV or more and 150 kV or less, and the phosphor may emit light of red area including a bright line spectrum in the wavelength range of 600 nm or more and 630 nm or less. In this dosimeter, it is capable of suppressing influence on an image taken or fluoroscopically viewed by using an X-ray which is emitted from the X-ray generating apparatus with the foregoing predetermined range of tube voltage and has energy and species of radiation suitable for medical image diagnosis, and measuring a dose of the X-ray in real time during taking or fluoroscopically viewing the X-ray image. Furthermore, the light emitted from the phosphor when receiving the X-ray has a bright line spectrum in the wavelength range of 600 nm or more and 630 nm or less corresponding to a transmitting wavelength range of an easily available optical fiber. Accordingly, it is capable of providing a dosimeter with low price, by which sensitivity and accuracy for the dose of X-ray to be measured can be enhanced.

In the foregoing dosimeter, an end surface of a light incident side part of the optical fiber may be an inclined surface inclined with respect to the optical axis of the optical fiber and the phosphor of the radiation detection part may be disposed to face a peripheral surface in opposition to the inclined surface of the optical fiber so that the light emitted from the phosphor enters from the peripheral surface and reaches the inclined surface. In this dosimeter, the light emitted from the phosphor of the radiation detection part is condensed so as to direct to a core around the optical axis of the optical fiber due to refraction on the peripheral surface, when entering from the peripheral surface in opposition to the inclined surface of the optical fiber. The light passing through the inside of the optical fiber while being condensed in this way reaches the inclined surface from inside of the optical fiber, and is reflected at the inclined surface and transmitted in the core around the optical axis so as to be directed to a light-outgoing end part. By entering the light from the peripheral surface of the optical fiber and reflecting the light from the inside at the inclined surface in this way, it is capable of efficiently guiding and transmitting the light, which is emitted from the phosphor, in the core of optical fiber, in comparison with a case of directly entering the light from outside into the end surface of light-incoming end part of the optical fiber. Accordingly, it is capable of further improving sensitivity and accuracy for the dose of radiation to be measured.

In the foregoing dosimeter, the inclined surface of the light-incoming end part of the optical fiber may be mirror finished and applied with a light reflective coating to enhance reflectivity to the foregoing light. In this dosimeter, since the inclined surface of the optical fiber is mirror finished, light scattering at the inclined surface can be reduced. Moreover, since the inclined surface is applied with the light reflective coating to enhance light reflectivity, it is capable of enhancing the reflectivity of light when the light entered from the peripheral surface of the optical fiber and transmitted inside of the optical fiber is reflected at the inclined surface. Accordingly, it is capable of further efficiently guiding and transmitting the light, which is emitted from the phosphor, in the core of optical fiber and further improving the sensitivity and accuracy of the dose of radiation to be measured.

In the foregoing dosimeter, the foregoing optical fiber may be an optical fiber made of a fluororesin. In this dosimeter, by using the optical fiber made of a fluororesin, it is capable of having a satisfactory transparency to an X-ray and transmitting a red light emitted from the phosphor made of Y₂O₂S as a matrix with at least Eu as an activating agent, with low transmission loss.

### Advantageous Effects of Invention

According to the present invention, it is capable of providing a dosimeter with an environmental safety, by which a dose of radiation can be measured in real time when taking or fluoroscopically viewing an image by using the radiation while suppressing influence on the image taken or fluoroscopically viewed.

### BRIEF DESCRIPTION OF DRAWINGS

FIG. 1 is a schematic diagram showing one configuration example of an overall configuration of a dosimeter according to an embodiment of the present invention.

FIG. 2 is a schematic diagram showing another configuration example of an overall configuration of a dosimeter according to an embodiment of the present invention.

FIG. 3 is an enlarged view of one configuration example of an X-ray detection part in the dosimeter of the present embodiment.

FIG. 4 is an enlarged view of one configuration example of a light-incoming end part of an optical fiber in the dosimeter of the present embodiment.

FIG. 5A is a side view of the light-incoming end part in the view from a direction perpendicular to the optical axis of the optical fiber, and FIG. 5B is a front view of the light-incoming end part in the view from the optical axis of the optical fiber.

FIG. 6 is a schematic diagram showing one example of a main body apparatus of the dosimeter of the present embodiment.

FIG. 7 is an illustration showing a state of measuring an X-ray dose in real time when taking or fluoroscopically viewing an X-ray image for medical image diagnosis by using the dosimeter of the present embodiment.

### DESCRIPTION OF EMBODIMENTS

Hereinafter, embodiments of the present invention will be described with reference to the drawings. It is noted that, although the present embodiment will be described with respect to an example in which the present invention is applied to a dosimeter of measuring a dose of X-ray, the present invention is applicable to a dosimeter of measuring a dose of radiation except for an X-ray.

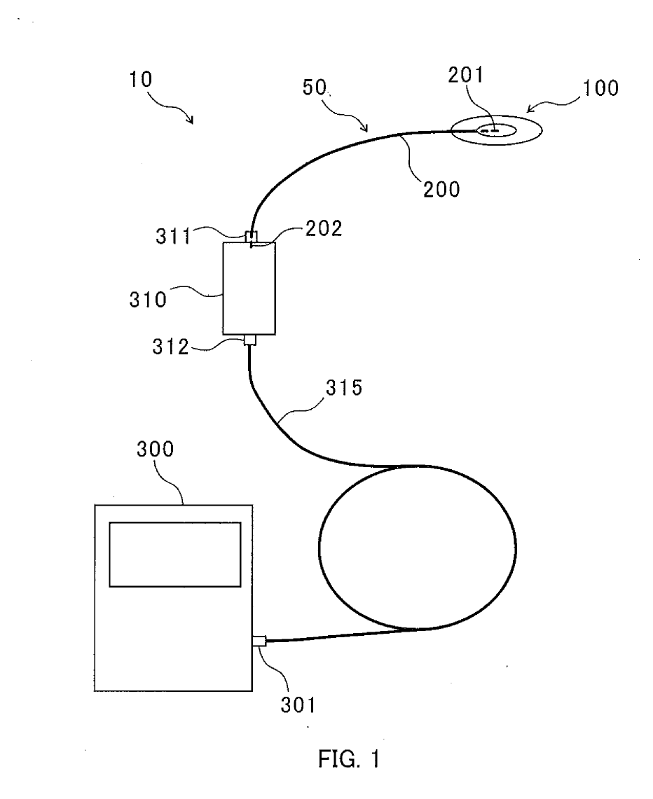

FIG. 1 is a schematic diagram showing one configuration example of an overall configuration of a dosimeter according to an embodiment of the present invention. In FIG. 1, a dosimeter 10 of the present embodiment includes an X-ray detection part 100 as a radiation detection part, an optical fiber 200 as an optical transmission body, a main body apparatus 300 and a light detection part 310. The X-ray detection part 100 and optical fiber 200 are integrally configured so as to be a set of X-ray detection probe 50. Two or more sets of X-ray detection probe 50 may be included.

The X-ray detection part 100 has a phosphor that emits light by receiving an X-ray as a radiation.

The optical fiber 200 has a light-incoming end part 201 where the light emitted from the phosphor of the X-ray detection part 100 is entered and a light-outgoing end part 202 where the light entered and transmitted from the light-incoming end part 201 exits. By using the optical fiber 200, since the light that is emitted from the phosphor of X-ray detection part 100 when receiving an X-ray can be transmitted to the light detection part 310 away from the X-ray detection part 100, the X-ray is not blocked by the light detection part 310.

Furtheremore, the light detection part 310 detects the light that exits from the light-outgoing end part 202 of the optical fiber 200. The light detection part 310 includes an optical fiber connection part 311 that is detachably connected with the light-outgoing end part 202 of the optical fiber 200, and a cable connection part 312 that is detachably connected with an end of a cable 315. Another end of the cable 315 is detachably connected with a cable connection part 301 of the main body apparatus 300. The cable 315 has a function of transmitting a signal detected with the light detection part 310 to the main body apparatus 300. As the light detection part 310, for example, a photomultiplier tube (PMT), a photodiode, or the like can be used. It is noted that electric power required for the light detection part 310 may be supplied from a battery, which is installed in the light detection part 310, or may be supplied from the main body apparatus 300 side via the cable 315.

Moreover, the light detection part 310 may be configured so that two or more sets of the X-ray detection probes 50 can be detachably connected therewith. In the example of FIG. 1, although it is shown a case in which the number of optical fiber connection part 311 is one, multiple (for example, two, three or more) optical fiber connection parts 311 may be provided so that two or more X-ray detection probes 50 can be connected simultaneously. The main body apparatus 300 may include two or more cable connection parts 301 so as to be connected with two or more light detection parts 310.

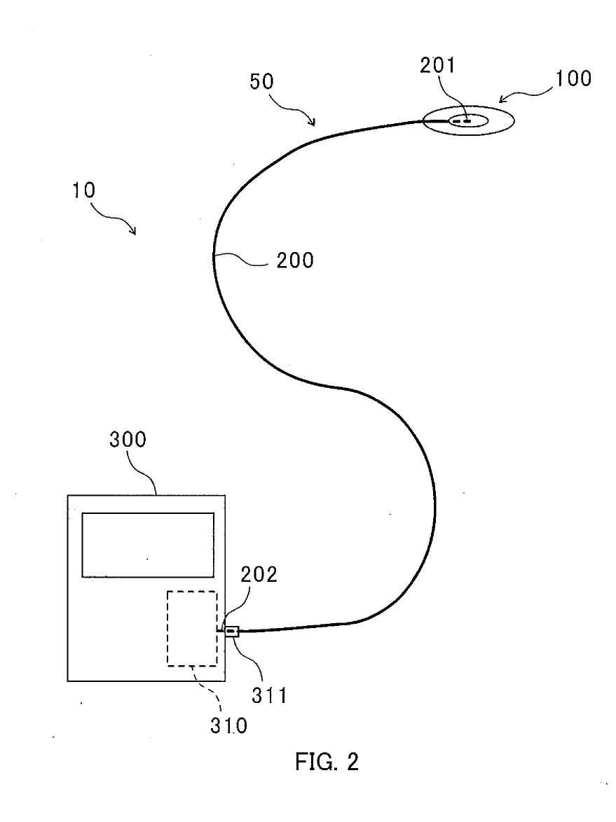

FIG. 2 is a schematic diagram showing another configuration example of an overall configuration of a dosimeter according to an embodiment of the present invention. The dosimeter 10 in FIG. 2 is an example in which the light detection part 310 is incorporated in the main body apparatus 300. In the case of this configuration example, the light-outgoing end part 202 of the optical fiber 200 is detachably connected with the optical fiber connection part 311 of the main body apparatus 300. In the example of FIG. 2, although it is shown a case in which the number of optical fiber connection part 311 is one, multiple (for example, two, three or more) optical fiber connection parts 311 may be provided so that two or more X-ray detection probes 50 can be connected simultaneously. In the case the multiple optical fiber connection parts are provided, two or more light detection part 310 may be provided corresponding to each of the multiple optical fiber connection parts. A single light detection part 310 may be shared so as to detect light from each of the multiple optical fiber connection parts while switching over the optical fiber connection parts.

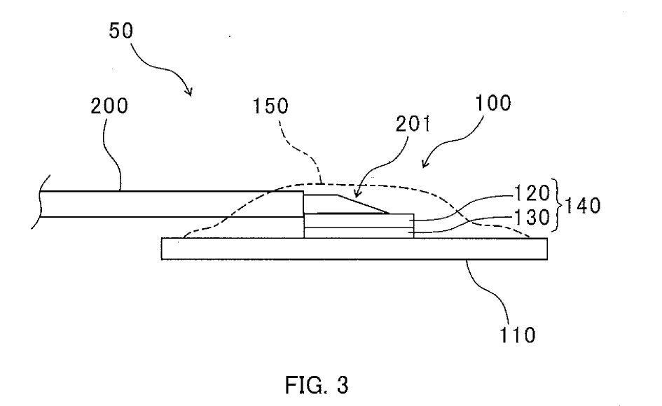

FIG. 3 is an illustration of one configuration example of the X-ray detection part 100 in the dosimeter 10 of the present embodiment.

The X-ray detection part 100 includes a base member 110 made of, for example, plastic material, and a phosphor sheet 140 provided on the base member 110 in the form of a layer or a plate. The phosphor sheet 140, for example, has a two-layered structure and includes a phosphor 120 and a support 130 of supporting the phosphor 120.

As the support 130, for example, a support having the shape of a thin plate, which is made of plastic material such as acrylic, polyethylene, etc. can be used. The thickness of the support 130 is, for example, in the range of 0.05-1.0 [mm] and preferably in the range of 0.1-0.5 [mm]. It is noted that, with respect to the material and thickness of the support 130, it is enough to have a satisfactory transparency to an X-ray to the extent that influence on an image taken or fluoroscopically viewed by using the X-ray can be suppressed, and it is not limited to the foregoing examples.

The mass per unit area of the layer-shaped phosphor 120 coated and dried on a surface (upper surface in the figure) perpendicular with respect to the thickness direction of the support 130 is, for example, in the range of 20-400 [mg/cm²] and preferably in the range of 100-300 [mg/cm²]. The thickness of the phosphor 120 is, for example, in the range of 0.5-1.5 [mm] and preferably in the range of 0.8-0.9 [mm]. It is noted that, with respect to the quantity and thickness of the phosphor 120, it is enough to acquire a satisfactory fluorescent light necessary for taking or fluoroscopically viewing an image by using an X-ray and have a satisfactory transparency to the X-ray to the extent that influence on an image taken or fluoroscopically viewed by using the X-ray can be suppressed, and it is not limited to the foregoing examples.

There are several methods to forming the foregoing phosphor sheet 140 and the phosphor sheet can be formed, for example, by using following methods. To start with, binder with organic synthetic resin dissolved in organic solvent, etc. is added to phosphor powder, and coating liquid like paint slurry with the phosphor turbidly mixed in the binder is prepared. By coating this coating liquid on the support 130 so as to have a predetermined coated mass and drying it, the phosphor sheet 140 having the phosphor 120 of the foregoing mass per unit area can be acquired. In coating of the foregoing coating liquid, in addition to a method with a brush or spray used in painting, various kinds of a coating tool, coating machine or printing machine, which is called a coater used in printing or the like, may be used. The coated film may be dried by heating in addition to drying at room temperature. The phosphor sheet 140 may be formed by a method except for the foregoing exemplified method.

The phosphor sheet 140 with the foregoing configuration is, for example, fixed on the base member 110 with an adhesive material so that the side near the support 130 is attached to the base member 110. On the surface (lower surface in the figure) of the base member 110, which is opposite to the phosphor sheet 140, a layer of adhesive may be formed so as to easily attach on a skin of a human body and so on. The shape in a surface direction perpendicular to a thickness direction of the base member 110, on which the phosphor sheet 140 is formed, may be, for example, a square with the sides of about several mm to ten and several mm, or a circular shape with a diameter of about several mm to ten and several mm. Each of these surfaces may have a planar shape or a curved surface shape.

The light-incoming end part 201 of the optical fiber 200 is fixed on the phosphor sheet 140 formed on the base member 110 with, for example, an adhesive material. As shown with a dashed line in FIG. 3, a light-shielding cover part 150, which blocks light by entirely covering the phosphor sheet 140 and the light-incoming end part 201 of the optical fiber 200, is formed. The light-shielding cover part 150 also has a function of protecting the phosphor sheet 140 formed on the base member 110 and the light-incoming end part 201 of the optical fiber 200, and a function of preventing the deviation of positioning relation between the phosphor sheet 140 and the light-incoming end part 201 of the optical fiber 200 with more certainty. The light-shielding cover part 150 may be formed, for example, with the foregoing adhesive material for fixing. The light-shielding cover part 150 may be formed with other resin except for the foregoing adhesive material so as to entirely cover the phosphor sheet 140 and the light-incoming end part 201 of the optical fiber 200 after fixing the light-incoming end part 201 of the optical fiber 200 with the adhesive material.

The phosphor 120 is a phosphor made of Y₂O₂S as a matrix with at least Eu as an activating agent and emits light by receiving an X-ray as a radiation. In this embodiment, a phosphor made of Y₂O₂S:Eu,Sm, in which a small amount of Sm for improving characteristics is further added, is used as the phosphor 120. It is noted that a phosphor made of Y₂O₂S:Eu without Sm added may be used as the phosphor 120.

The phosphor 120 made of the foregoing predetermined material emits light in a red region, which has a bright line spectrum in the wavelength range of 600 nm or more and 630 nm or less, when receiving an X-ray from an X-ray generating apparatus in which, for example, a target of tungsten, molybdenum or the like is used and a tube voltage is set to 40 kV or more and 150 kV or less. The wavelength range of 600 nm or more and 630 nm or less corresponds to a transmittable wavelength range of an easily available optical fiber. Since the phosphor 120 made of the foregoing predetermined material does not include cadmium (Cd), it is capable of configuring the dosimeter that includes the X-ray detection part 100 having an environmental safety.

The foregoing phosphor 120 made of Y₂O₂S as a matrix with at least Eu as an activating agent has a satisfactory transparency to an X-ray. For example, when receiving an X-ray from the aforementioned X-ray generating apparatus in which a target of tungsten, molybdenum or the like is used and a tube voltage is set to 40 kV or more and 150 kV or less, the absorbance A of the phosphor 120 to the X-ray is 1.3 or less. When the absorbance A is 1.3 or less like this, an image of the phosphor 120 does not appear in the X-ray image taken or fluoroscopically viewed, or there is no influence of the image of the phosphor 120 on a treatment and diagnosis using the X-ray image taken or fluoroscopically viewed even when the image of the phosphor 120 appears on the X-ray image. It is noted that, when the intensity of X-ray made incident on the phosphor 120 is I₀ and the intensity of X-ray passing through the phosphor 120 is I, the absorbance A is defined by the following formula (1).

A=−log₁₀(I/I₀) (1)

The foregoing phosphor 120 made of Y₂O₂S as a matrix with at least Eu as an activating agent has small degradation in brightness due to damage (radiation damage) when an X-ray is irradiated. For example, when an X-ray from the foregoing X-ray generating apparatus is irradiated to the phosphor 120 in the present embodiment so that the accumulated absorption dose become 2 [Gy], the degradation in brightness of luminescence from the phosphor 120 after irradiation is within 10% of the brightness before irradiation.

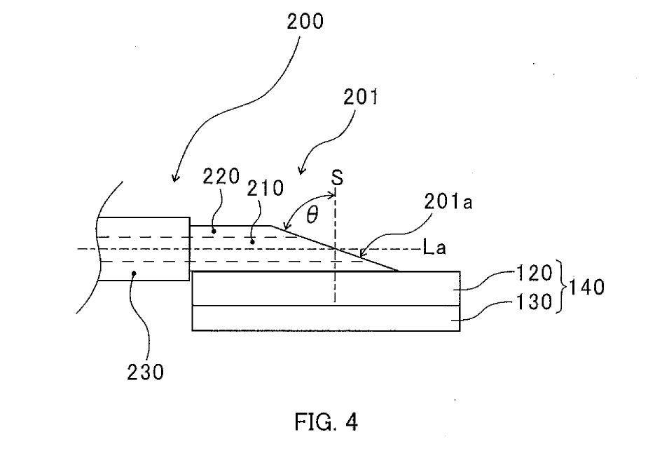

FIG. 4 is an enlarged view of one configuration example of the light-incoming end part 201 of the optical fiber 200 in the dosimeter 10 of the present embodiment.

The optical fiber 200 in the present embodiment is a step-index type optical fiber including a core 210 that forms the portion around the optical axis (central axis) and a clad 220 that is formed so as to surround the core 210. The outer surface (peripheral surface) of the clad 220 is protected with a coating 230. At the boundary between the core 210 and clad 220 of the optical fiber 200, a refractive index changes stepwise and the core 210 has a higher refractive index than the clad 220. Light entered from the light-incoming end part 201 of the optical fiber 200 passes mainly in the core 210 and is transmitted toward the light-outgoing end part 202. It is noted that a graded index type optical fiber formed so that a refractive index changes continuously from the core to the clad, may be used as the optical fiber.

The material of the optical fiber 200 preferably has a satisfactory transparency to an X-ray and is preferably capable of transmitting red light in the wavelength range of 600 nm or more and 630 nm or less, which is emitted from the phosphor 120, with low transmission loss. As such an optical fiber, an optical fiber made of acrylic resin such as polymethyl methacrylate (PMMA), etc., and an optical fiber made of fluororesin can be exemplified. The optical fiber 200 made of such material also has a satisfactory transparency to an X-ray, unlike a cable and lead wire made of normal metal. Particularly, the optical fiber made of fluororesin is preferably capable of transmitting red light emitted from the phosphor 120 with lower transmission loss compared to the optical fiber made of acrylic resin such as PMMA and so on, since the optical fiber 200 made of such material does not have an absorption peak in the wavelength range of 600 nm or more and 630 nm or less.

The end surface of the light-incoming end part 201 of the optical fiber 200 is an inclined surface 201a inclined by a predetermined angle θ with respect to a virtual plane S perpendicular to the optical axis La of the optical fiber 200. The inclination angle θ of the inclined surface 201a is, for example, an angle in the range of 30 degrees-60 degrees, and more preferably, an angle in the range of 40 degrees-50 degrees. The inclined surface 201a of the optical fiber 200 is mirror polished to reduce scattering of light emitted from the phosphor 120. A light reflective coating (for example, silver color coating) to enhance reflectivity to the light emitted from the phosphor 120 is applied.

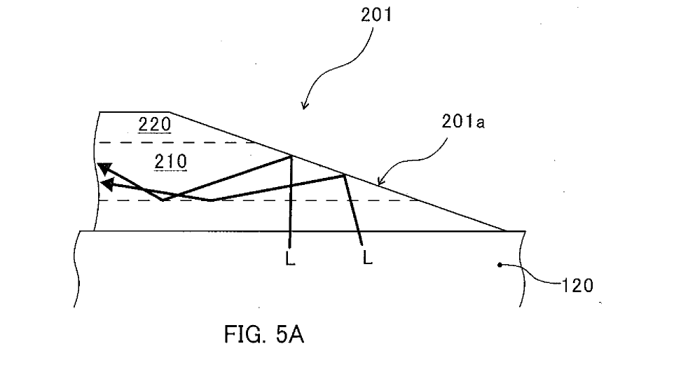

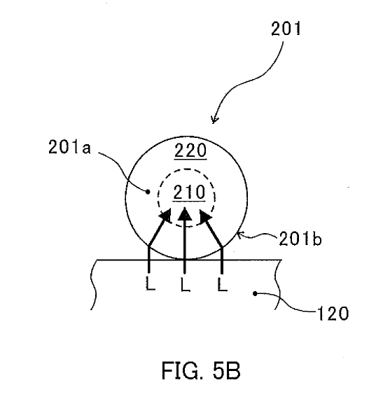

At the light-incoming end part 201 of the optical fiber 200, the coating 230 is removed and the peripheral surface 201b of the clad 220 is exposed. The phosphor 120 of the X-ray detection part 100 is disposed in opposition to the peripheral surface 201b so that the light L emitted from the phosphor 120 enters from the peripheral surface 201b of the clad 220 in opposition to the inclined surface 201a of the optical fiber 200 and reaches the inclined surface 201a. By entering the light L from the peripheral surface 201b of the optical fiber 200 and reflecting the light L from the inside at the inclined surface 201a in this way, an incident efficiency of the light L to the optical fiber 200 is enhanced. The aspect where the incident efficiency of the light L to the optical fiber 200 is enhanced in the configuration of FIG. 4 as described above is obtained according to experiments and considerations by the present inventors. Although a mechanism that the incident efficiency of the light L to the optical fiber 200 is enhanced in the configuration of FIG. 3 is not clear, it is considered that, for example, a light collection function in light incident passes as shown in FIGS. 5A and 5B is also related to the mechanism.

FIGS. 5A and 5B are illustrations showing one example of a state that the light emitted from the phosphor 120 of the X-ray detection part 100 is guided in the optical fiber 200. FIG. 5A is a side view of the light-incoming end part 201 in the view from a direction perpendicular to the optical axis of the optical fiber 200 (from the front side in FIG. 4). FIG. 5B is a front view of the light-incoming end part 201 in the view from the optical axis of the optical fiber 200 (from the right side in FIG. 4).

In FIGS. 5A and 5B, an X-ray to be measured is entered from the upper side or lower side in the figures and passes through the phosphor 120 of the X-ray detection part 100. When entering from the peripheral surface 201b in opposition to the inclined surface 201a of the optical fiber 200, the light L emitted from the phosphor 120 of the X-ray detection part 100 is condensed so as to direct to the core 210 around the optical axis La of the optical fiber 200 due to refraction on the inclined surface 201b. The light L passed through inside of the optical fiber 200 while being condensed in this way reaches the inclined surface 201a from inside of the optical fiber 200, and is reflected at the inclined surface 201a and transmitted in the core 210 around the optical axis so as to be directed to a light-outgoing end part 202. By entering the light L from the peripheral surface 201b of the optical fiber 200 and reflecting the light L from the inside at the inclined surface 201a in this way, it is capable of enhancing an incident efficiency of the light L, and efficiently guiding and transmitting the light L emitted from the phosphor 120 in the core 210 of the optical fiber 200, in comparison with a case of directly entering the light from outside into the end surface of light-incoming end surface of the optical fiber 200. Accordingly, it is capable of further improving sensitivity and accuracy for the dose of X-ray to be measured.

It is noted that, although the light L emitted from the phosphor 120 is entered from the peripheral surface 201b side of optical fiber 200 in the present embodiment as described above, a configuration for entering the light L emitted from the phosphor 120 into the optical fiber 200 is not limited to the configuration in the present embodiment. For example, the phosphor 120 may be disposed so as to be attached or close to the exposed inclined surface 201a of the optical fiber 200 and the light L emitted from the phosphor 120 may be directly entered into the inclined surface 201a. The configuration such as the present embodiment, in which the light form the phosphor 120 is entered from the peripheral surface 201b side of optical fiber 200, may be combined with the configuration of directly entering the light from the phosphor 120 into the inclined surface 201a.

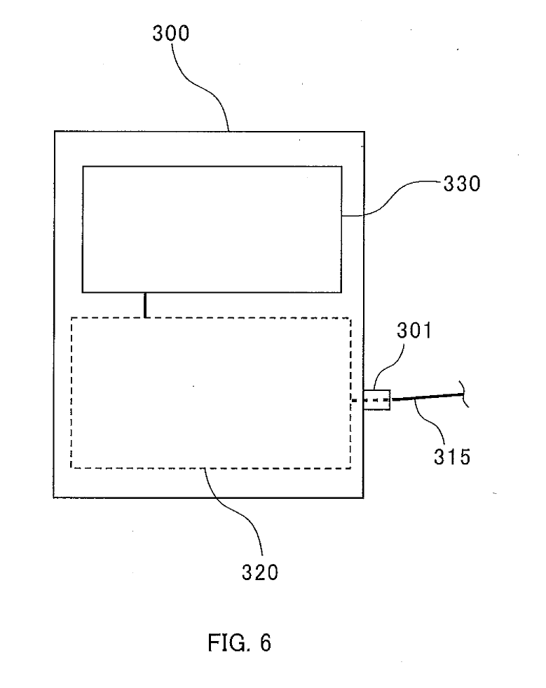

FIG. 6 is a schematic diagram showing one example of the main body apparatus 300 of the dosimeter of the present embodiment. FIG. 6 shows a configuration example of the main body apparatus 300 in a case that the overall configuration of dosimeter is the aforementioned configuration in FIG. 1. The main body apparatus 300 includes a cable connection part 301 that is connected with a cable 315 between the light detection part 310 and the main body apparatus 300. It is noted that, in a case that the overall configuration of dosimeter is the aforementioned configuration in FIG. 2, the main body apparatus 300 includes the foregoing optical fiber connection part 311 connected with the light-outgoing end part 202 of the optical fiber 200 which forms the X-ray detection probe 50, and the light detection part 310 detecting light that exits from the light-outgoing end part 202 of the optical fiber 200.

The main body apparatus 300 also includes a control part 320 which functions as control means and computation means, a display part 330 as output means of outputting a measurement result, and so on. The control part 320 is configured with a microcomputer including, for example, a CPU, a ROM, a RAM, an I/O interface, etc. and connected with each part such as the light detection part 310, the display part 330 and so on.

The control part 320 controls each part and calculates values of various doses of X-rays (for example, absorption dose [Gy], dose equivalent [Sv], exposure dose [C/kg]) or a value of dose rate [Gy/h] that is a dose per unit hour based on an output signal of the light detection part 310, by executing a predetermined program. The control part 320 also memorizes calibration data and values of various coefficients and parameters for calculating the foregoing various doses and dose rate. The calibration data is a conversion table or data of a conversion formula, which is used when converting the output signal of the light detection part 310 to values of the foregoing various doses and dose rate, and is acquired by a calibration performed before starting the use.

In a case that two or more X-ray detection probes 50 are used and the characteristics of X-ray detection probes 50 are different each other, two or more kinds of calibration data acquired for each of the X-ray detection probes 50 are memorized in advance. In this case, the control part 320 identifies the X-ray detection probe connected with the light detection part 310 or the main body apparatus 300 and calculates the foregoing various doses and dose rate by using the corresponding calibration data. Furtheremore, in a case that the main body apparatus 300 is connected with two or more light detection parts 310 or the main body apparatus 300 is provided with two or more light detection parts 310 and the characteristics of light detection parts 310 are different each other, two or more kinds of calibration data acquired for each of the light detection parts 310 or two or more kinds of calibration data acquired for each combination set of the X-ray detection probes 50 and the light detection parts 310 are memorized in advance. It is noted that, since the aforementioned calibration data possibly changes after starting the use, the acquisition and update may be periodically performed after starting the use.

The display part 330 is configured with, for example, a liquid crystal display or the like, which is capable of displaying a value of dose or dose rate calculated by the control part 320 in real time and displaying a change of dose or dose rate as a graph with time axis in real time.

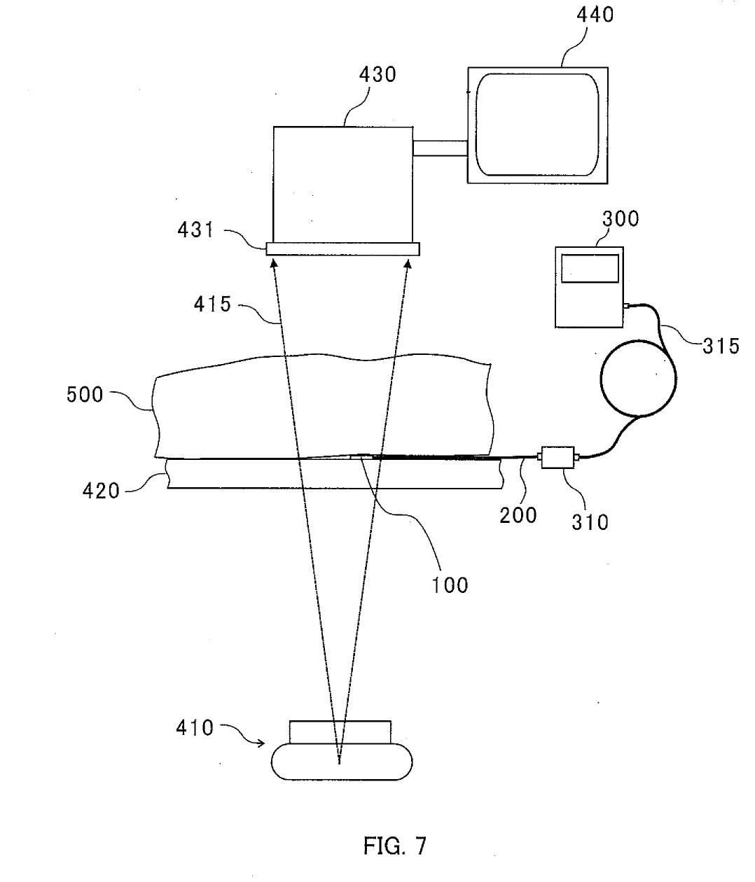

FIG. 7 is an illustration showing a state of measuring a skin exposure dose of X-rays in real time when taking or fluoroscopically viewing an X-ray image for medical image diagnosis by using the dosimeter 10 of the present embodiment. The example in FIG. 7 is an example in which a patient is treated by watching an X-ray image in real time with a method of medical treatment called an IVR (Interventional Radiology). The IVR generally means a “therapeutic application of radiological diagnosis technique”, which is also sometimes used as a synonym almost same as an “intravascular treatment”, an “intravascular surgery”, a “low invasive treatment”, an “image-guided therapy” and so on. The IVR is a method of medical treatment for curing a disease by inserting a small tube (catheter or needle) into a body while watching an image including a diseased part such as an X-ray image which is taken or fluoroscopically viewed, CT and so on in the present embodiment, and includes kinds of a vascular IVR and a non-vascular IVR. Since these kinds of IVR are performed while irradiating an X-ray, in order to prevent a skin exposure disorder of a patient, it is necessary to accurately grasp and manage an exposed dose on a skin surface of the patient (particularly a skin surface of X-ray incidence side having a high possibility of the occurrence of exposure disorder).

Accordingly, in the example in FIG. 7, the X-ray detection part 100 of dosimeter 10 of the present embodiment is pasted on a skin surface of the patient where an X-ray image is taken or fluoroscopically viewed, and the skin exposure dose at the position is displayed and recorded by measuring it in real time.

In FIG. 7, an X-ray 415 with a predetermined species and dose of radiation, which is generated from an X-ray generating apparatus (X-ray source) 410 having an X-ray tube to which the aforementioned tube voltage is applied, is irradiated to the predetermined portion of a human body 500 of patient on a catheter table 420. The X-ray 415 passed through the human body is used for taking or fluoroscopically viewing the X-ray image in real time by an X-ray fluoroscopic imaging apparatus 430 having an X-ray image intensifier 431. Herein, the X-ray image intensifier 431 is an apparatus for converting a two-dimensional X-ray image received on an incident phosphor surface to a visible light image and outputting the visible light image. It is noted that a flat panel detector (FPD) may be used instead of the X-ray image intensifier 431. The image taken or fluoroscopically viewed by the X-ray fluoroscopic imaging apparatus 430 is displayed on an image display apparatus 440 in real time and used for a medical treatment of the patient. In order to measure a skin exposure dose of the patient in the IVR with taking or fluoroscopically viewing such an the X-ray image, the X-ray detection part 100 of the dosimeter 10 in the present embodiment is pasted on a skin of lower side of the patient, for which the X-ray image is taken or fluoroscopically viewed, in the figure.

As described above, according to the present embodiments, when the X-ray detection part 100 receives an X-ray, the phosphor 120 included in the X-ray detection part 100 emits light, and the light emitted from the phosphor 120 of the X-ray detection part 100 enters from the light-incoming end part 201 of the optical fiber 200, transmits in the optical fiber 200 and exits from the light-outgoing end part 202. This light exited from the light-outgoing end part 202 of the optical fiber 200 is detected with the light detection part 310. Based on a result of the light detection part 310, a dose of X-ray can be measured.

Herein, the light that is emitted from the phosphor 120 of the X-ray detection part 100 by receiving the X-ray can be transmitted to the light detection part 310 away from the X-ray detection part 100 by the optical fiber 200. Accordingly, when measuring a dose of X-ray while taking or fluoroscopically viewing an X-ray image, it is not necessary to locate the light detection part 310 at the region where the X-ray for taking or fluoroscopically viewing is passing through and the X-ray for taking or fluoroscopically viewing is not blocked by the light detection part 310. Furtheremore, the foregoing phosphor 120 made of Y₂O₂S as a matrix with at least Eu as an activating agent has a satisfactory transparency to the X-ray and the optical fiber 200 has also a satisfactory transparency to the X-ray unlike a cable and lead wire made of normal metal. Accordingly, when taking or fluoroscopically viewing an image by using the X-ray, it is capable of suppressing an influence on the image taken or fluoroscopically viewed. Therefore, it is capable of measuring a dose of X-ray in real time when taking or fluoroscopically viewing the image while suppressing influence on the image taken or fluoroscopically viewed by using the X-ray.

Moreover, the foregoing phosphor 120 of the X-ray detection part 100, which is made of Y₂O₂S as a matrix with at least Eu as an activating agent, does not include cadmium (Cd). Accordingly, it is capable of providing a dosimeter with an environmental safety.

In particular, the dosimeter 10 of the present embodiment can measure a dose of X-ray in real time when taking or fluoroscopically viewing the X-ray image while suppressing influence on the image taken or fluoroscopically viewed by using the X-ray with energy and species suitable for medical image diagnosis, which is emitted from the X-ray generating apparatus with the predetermined range of tube voltage and has energy and species for medical image diagnosis. Accordingly, the dosimeter 10 of the present embodiment is preferable as a safe real-time dosimeter used for medical image diagnosis such as a real-time measurement of a skin exposure dose when performing an angiography, a vascular IVR, a non-vascular IVR, or the like.

Further, the dosimeter 10 of the present embodiment can also used as a dosimeter of measuring an exposure dose for a medical staff such as an IVR medical operator.

It is noted that the description of embodiments disclosed in the present specification is provided so that the present disclosures can be produced or used by those skilled in the art. Various modifications of the present disclosures will be readily apparent to those skilled in the art and general principles defined in the present specification can be applied to other variations without departing from the spirit and scope of the present disclosures. Therefore, the present disclosures should not be limited to examples and designs described in the present specification and should be recognized to be in the broadest scope corresponding to principles and novel features disclosed in the present specification.

### REFERENCE SIGNS LIST

10 dosimeter
50 X-ray detection probe
100 X-ray detection part
110 base member
120 phosphor
130 support
140 phosphor sheet
150 light-shielding cover part
200 optical fiber
201 light-incoming end part
202 light-outgoing end part
210 core
220 clad
230 coating
300 main body apparatus
301 cable connection part
310 light detection part
311 optical fiber connection part
312 cable connection part
315 cable
320 control part
330 display part
410 X-ray generating apparatus (X-ray source)
415 X-ray
420 catheter table
430 X-ray fluoroscopic imaging apparatus
431 X-ray image intensifier (I.I.) or flat panel detector (FPD)
440 image display apparatus
500 human body (patient)
L light

1. A dosimeter of measuring a dose of radiation, comprising:
a radiation detection part including a phosphor made of Y₂O₂S as a matrix with at least Eu as an activating agent;
an optical fiber of transmitting light emitted from the phosphor of the radiation detection part by receiving a radiation; and
a light detection part of detecting the light transmitted through the optical fiber.

2. The dosimeter according to claim 1,
wherein the radiation is an X-ray emitted from an X-ray generating apparatus with a tube voltage of 40 kV or more and 150 kV or less, and
wherein the phosphor emits light of red area including a bright line spectrum in the wavelength range of 600 nm or more and 630 nm or less, when receiving the X-ray.

3. The dosimeter according to claim 1,
wherein an end surface of a light incident side part of the optical fiber is an inclined surface inclined with respect to the optical axis of the optical fiber, and
wherein the phosphor of the radiation detection part is disposed to face a peripheral surface in opposition to the inclined surface of the optical fiber so that the light emitted from the phosphor enters from the peripheral surface and reaches the inclined surface.

4. The dosimeter according to claim 3, wherein the inclined surface of the optical fiber is mirror polished and applied with a light reflective coating to enhance reflectivity to the light.

5. The dosimeter according to claim 1, wherein the optical fiber is an optical fiber made of a fluororesin.

6. The dosimeter according to claim 2,
wherein an end surface of a light incident side part of the optical fiber is an inclined surface inclined with respect to the optical axis of the optical fiber, and
wherein the phosphor of the radiation detection part is disposed to face a peripheral surface in opposition to the inclined surface of the optical fiber so that the light emitted from the phosphor enters from the peripheral surface and reaches the inclined surface.

7. The dosimeter according to claim 2, wherein the optical fiber is an optical fiber made of a fluororesin.

8. The dosimeter according to claim 3, wherein the optical fiber is an optical fiber made of a fluororesin.

9. The dosimeter according to claim 4, wherein the optical fiber is an optical fiber made of a fluororesin.

10. The dosimeter according to claim 6, wherein the optical fiber is an optical fiber made of a fluororesin.
